(19) United States
(12) Patent Application Publication
(10) Pub. No.: US 2004/0156840 A1
(43) Pub. Date: Aug. 12, 2004

(54) Use of igm antibodies against dsdna in systemic lupus erythematosus with nephritis

(76) Inventors: Torsten Witte, Hannover (DE); Torsten Matthias, Flonheim (DE)

Correspondence Address: Kevin S. Lemack, Nields & Lemack, Suite 7, 176 E Main Street, Westboro, MA, 01581, US

(21) Appl. No.: 10/479,939

(22) PCT Filed: Jun. 7, 2002
(86) PCT No.: PCT/DE02/02085

(30) Foreign Application Priority Data
Jun. 7, 2001 (DE) 101 27 712.1

(51) Int. Cl. A61K 39/395

(52) U.S. Cl. 424/130.1

(57) ABSTRACT

A method for the production of a means for treating or preventing systemic lupus erythematosus is disclosed. Said means comprises IgM for sub-cutaneous and/or intra-muscular application. A preferred IgM is autoantigen IgM, in particular anti-ds-DNA and anti-phospholipid IgM. A pharmaceutical combination package for treatment of SLE is also disclosed, containing the IgM antibodies, together with at least one anti-inflammatory agent, at least one immuno-suppressive agent and/or at least one cytostatic agent for simultaneous and/or serial application.

### DESCRIPTION:

The invention concerns the use of immunoglobulins for the treatment of systemic lupus erythematosus.

Lupus erythematosus (SLE) involves an extremely difficult, increasingly serious disorder, which leads to death in many cases. SLE is an autoimmune disorder, in which antibodies to the body's own intracellular components are formed, which become apparent, e.g., as tissue lesions made up of self-contained cells, which are viewed as foreign to the body by the immune system and are thus attacked. A fatal reaction cycle arises in this way. SLE occurs for the most part and for the first time in young women. The incidence in the total population amounts to approximately 1 in 2000, but in women between 20 and 30 years old, approximately 1 in 700. In the course of the chronic disorder, in approximately half of the patients, a glomerulonephritis develops, which was earlier the most frequent cause of death due to SLE.

In addition to the characteristic so-called “butterfly” erythema, serious joint inflammations frequently occur during the long-lasting course of the disease.

Recurring pleurisy with or without effusion is also frequent. Serious SLE disorders are usually treated with an immediate corticosteroid therapy in order to attenuate resulting harmful sequelae such as hemolytic anemia, kidney damage, vasculitis, as well as acute CNS involvement. These therapies, however, frequently do not bring about satisfactory results in the serious stage of this life-threatening disorder.

This generalized autoimmune disorder of unexplained etiology is usually accompanied by the formation of numerous autoantibodies, immune complexes and changes in the complement system. Common clinical complaints are arthritis, skin inflammations such as erythema, changes in blood counts, nephritis, pleurisy, pericarditis and endocarditis, such as Libmann-Sacks disease, as well as neurologic and psychic disturbances. The course of the disease is variable and often ends in death after decades of chronic course.

Attempts have also been made to treat this disorder with nonsteroid antiphlogistics and immunosuppressive agents as well as with strong cytostatic agents. Thus, for example, T. Witte et al. describe a bolus therapy with cyclophosphamide for the treatment of lupus nephritis in “Deutsche Medizinische Wochenschrift” 118 (1993), 1005-1010. Lupus nephritis is one of the most serious organ complications of SLE due to the danger of kidney failure. Cyclophosphamide application, however leads to serious side effects, as with all cytostatic agents.

H. A. Austin et al., “New Engi J Med” 1986; 314: 614-9 show that for treatment of lupus glomerulonephritis, the intravenous administration of cyclophosphamide is superior to treatment with corticosteroids, such as prednisone, with respect to survival rate. It is possible by means of cyclophosphamide to attain a complete remission or at least a partial remission in the majority of patients.

In order to enable as accurate a diagnosis as possible of systemic lupus erythematosus, attempts have been made to find a correlation between actual laboratory parameters and clinical manifestation of the disorder. Thus T. Witte et al. in “Rheumatol Int” (1998) 18:85-91 describe the elevated presence of IgM anti-dsDNA antibodies in the serum of SLE patients. IgM anti-dsDNA antibodies were found in 52.3% of the cases. It was shown in this way that there exists a negative correlation between the emergence of nephritis and kidney failure and the presence of IgM anti-dsDNA antibodies, for which reason the authors assume that IgM anti-dsDNA antibodies provide a protection against the development of lupus nephritis.

R. Rieben et al. describe in “Blood”, Vol. 93, (1999) 942-951 that the addition of intravenous immunoglobulin preparations block complement activation and thus show an anti-inflammatory action. Therefore, human serum was incubated with serial dilutions of IV IgG and IV IgM and the formation of complement was determined by means of an ELISA assay. A dose-dependent inhibition of complement action was shown, for which reason, it is assumed that an intravenous administration of an IgM-enriched preparation could be used as complementary therapy to an anti-inflammatory treatment. It has been shown, however, that the intravenous administration of IgM antibodies shows no clinical effect.

Attempts have already been made to apply IgM intraperitoneally for the treatment of lupus erythematosus. This administration has also shown no effect (Boes M. et al., Proc. Nati. Acad. Sci. USA (2000) 97: 1184-9).

The object of the invention is thus to provide an agent for the treatment of systemic lupus erythematosus, which is effective and which does not have the above-described serious side effects.

This object is achieved by the features defined in the claims.

Namely, it has been found surprisingly that-although the intravenous administration of IgM antibodies has proven ineffective-the administration of such antibodies is extremely effective, however, when these are applied subcutaneously or intramuscularly. The invention thus concerns the use of immunoglobulins of the M class for the production of an agent for the treatment and/or prevention of systemic lupus erythematosus with subcutaneous and/or intramuscular application. Preferably, those IgM antibodies that are directed against the body's auto-antigens are used for this purpose. The body's auto-antigens are here intracellular antigens, particularly those in the cell nucleus and/or cycloplasm, such as double-stranded DNA as well as phospholipids, particularly cardiolipin.

It has been shown that IgM can be administered in any form without loss of effect, as long as they are administered s.c. and/or i.m. Usually, however, they are administered in the form of a solution, whereby encapsulated solutions or even encapsulated pure protein powder are particularly preferred as a slow-release formulation. The production of such slow-release formulations is generally known and comprises, for example, inclusion in fine biologically decomposable acrylate capsules or incorporation in a biologically decomposable three-dimensional network, for example, made up of collagen fibers, such as is known as so-called catgut. Such slow-release matrices can be deposited by means of a relatively thick needle without anything further into the subcutaneous tissue, for example, into an abdominal fold. It is possible in this way to offer a quantity of IgM that is effective over any desired period of time and a continuous serum level of IgM is maintained.

The pharmaceutical agent produced according to the invention is particularly suitable also for complementary therapy together with other known agents for the treatment of SLE and, in fact, particularly anti-inflammatory agents such as aspirin and/or corticosteroids as well as with cytostatic agents, particularly cyclophosphamide, azathioprine or mycophenolate mofetil.

The invention thus also concerns a pharmaceutical combination package for the simultaneous or time-displaced administration of an IgM-containing agent produced according to the invention together with another anti-inflammatory active ingredient, an immunosuppressive active ingredient and/or a cytostatic agent. It is possible to treat a plurality of clinical disease patterns of lupus erythematosus with the therapeutic agent produced according to the invention, such as, for example, nephritis, particularly glomerulonephritis, inclusive of a persistent proteinuria as well as acute and chronic kidney failure, as well as other organ manifestations, such as butterfly erythema, ulcers of the mucous membranes, polyarthritis, particularly non-deforming polyarthritis, arthralgias, hemarthrosis, serosites, such as pleuritis and pericarditis.

Treatment by means of plasmapheresis, i.e., an exchange of plasma with the removal of autoantibodies via an apparatus (immunopheresis), can also be combined with the administration of the agent produced according to the invention.

The invention will be explained in more detail with the following example.

So-called NZB×NZW (B/W) mice were used as the model system. Such mice are one of the standard animal models of SLE, since they typically develop autoantibodies and spontaneously develop lupus nephritis after 6-8 months. Four treatment groups, each containing 10 mice, were formed. These mice received the following therapies:

A: Control group, did not receive therapy

B: This group received intravenously in each animal 100 μg of a monoclonal HB 8329 antibody, starting from the age of 4 months at two-week intervals.

C: This group received a subcutaneous injection in each animal of 100 μg of the monoclonal antibody HB 8329 starting from the age of four months at weekly intervals.

D: This group received an IV injection in each animal of 100 μg of the monoclonal antibody HB 8329 starting from the age of seven months, i.e., after the beginning of nephritis at two-week intervals.

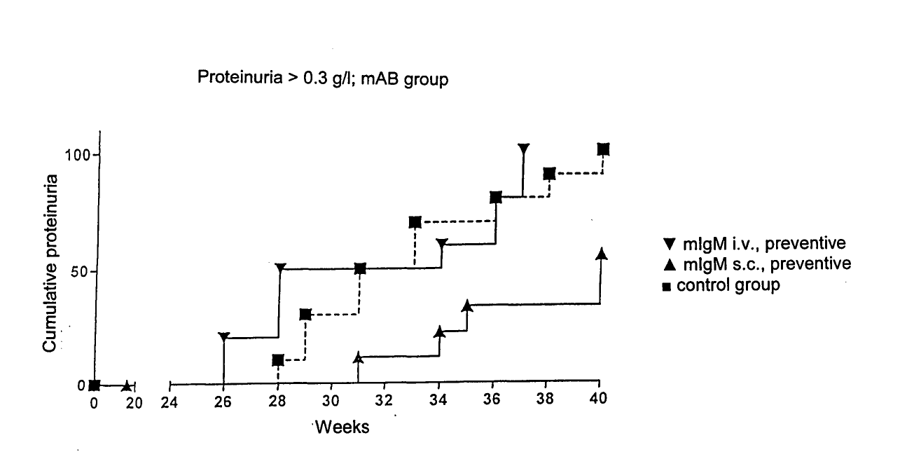

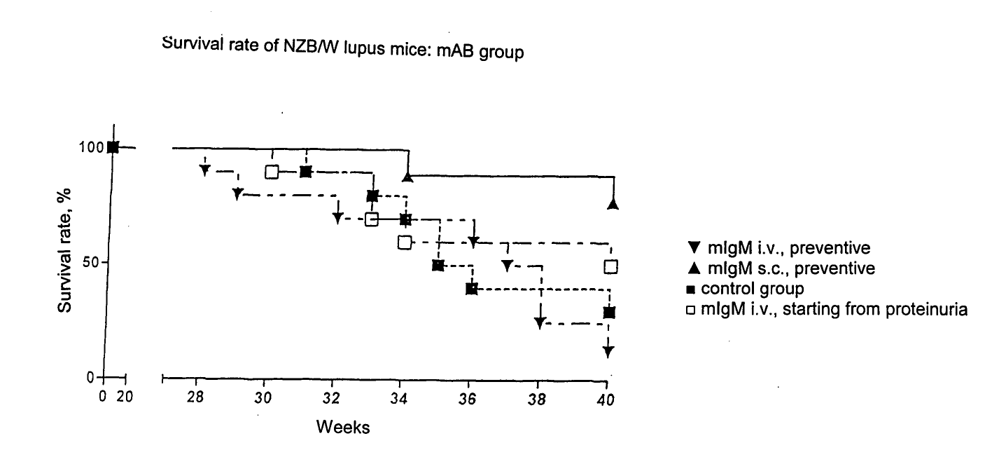

The antibodies were isolated from the culture supernatant of the hybridoma HB 8329 (ATCC) and purified as follows: The hybridoma HB 8329 was cultured in RPMI 1640 with an addition of penicillin/streptomycin and ITS medium without FCS. The culture supernatant was applied onto a Supertex 200 column (Pharmacia & Upjohn) and the large IgM molecule was separated from all other components of the culture medium. The results are shown in FIG. 1 and FIG. 2.

Here,

FIG. 1 shows the increase or the emergence of proteinuria and FIG. 2 shows the survival rate of the experimental animals.

As can be seen from FIG. 1, the course of cumulative proteinuria in the case of preventive intravenous administration of immunoglobulin M roughly corresponds to that of the untreated control group. This means that the intravenous administration of immunoglobulin M does not show a protective effect. In contrast to this, the subcutaneous application of IgM shows a clearly later onset of the disorder as well as a clearly slower course.

It is shown in FIG. 2 that the intravenous administration starting from the emerging proteinuria as well as the intravenous preventive administration shows no effect on the survival of the mice within the scope of statistical distribution. The death rate after 40 weeks in the control group lies at 20-50%, for both the preventive as well as the acute intravenous application, while in contrast, the survival rate lies at approximately 80-85% for the subcutaneous preventive application. This also substantiates the surprising and outstanding effect of the use of immunoglobulins of the M class according to the invention.

1. Use of immunoglobulin M for the production of an agent for the treatment and/or prevention of systemic lupus erythematosus with subcutaneous and/or intramuscular application.

2. Use according to claim 1, further characterized in that an autoantigen-IgM is used.

3. Use according to one of the preceding claims, further characterized in that the autoantigen is an antinuclear and/or anti-phospholipid antibody.

4. Use according to one of the preceding claims, further characterized in that the IgM is an anti-dsDNA [antibody].

5. Use according to one of the preceding claims, further characterized in that the IgM is an anti-cardiolipin IgM.

6. Use according to one of the preceding claims, further characterized in that the agent contains IgM in solution, encapsulated, as a slow-release formulation or as a pure substance.

7. Use according to one of the preceding claims, further characterized in that the agent is incorporated in an enclosed network made up of decomposable collagen fibers.

8. Use according to one of the preceding claims, further characterized in that the IgM is enclosed in a mixture of networks, which have a different dissolution rate.

9. A pharmaceutical combination for the treatment and/or prevention of systemic lupus erythematosus, containing an IgM as well as at least one anti-inflammatory agent, at least one immunsuppressive agent and/or at least one cytostatic agent for simultaneous and/or time-displaced application.

10. The pharmaceutical combination according to claim 9, further characterized in that the antiinflammatory agent is acetylsalicylic acid and/or a corticosteroid.

11. The pharmaceutical combination according to claim 9 or 10, further characterized in that the cytostatic agent is cyclophosphamide and/or azathioprine, methotrexate and/or mycophenolate mofetil.

12. The pharmaceutical combination according to one of claims 9-11, further characterized in that the immunosuppressive agent is cyclbsporin, leflunomide and/or a monoclonal or polyclonal antilymphocyte antibody.
